(19) United States
(12) Patent Application Publication
(10) Pub. No.: US 2010/0129432 A1
(43) Pub. Date: May 27, 2010

(54) MICROORGANISM-KILLING COMBINATION

(75) Inventors: Chin-Tin Chen, Taipei (TW); Tsuimin Tsai, Taipei (TW); Hsiung-Fei Chien, Taipei (TW); Tse-Hsien Wang, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: TAIPEI MEDICAL UNIVERSITY, Taipei (TW)

(21) Appl. No.: 12/277,772

(22) Filed: Nov. 25, 2008

(51) Int. Cl. A61K 9/127; A01N 43/04; A01P 1/00

(52) U.S. Cl. 424/450; 514/55

(57) ABSTRACT

The invention is directed to a microorganism-killing combination comprising a photosensitizer in an amount effective to kill microorganisms in a photodynamic process and a chitosan in an amount effective to enhance the microorganism-killing effect of the photosensitizer in the photodynamic process. The invention also provides the methods of administering the synergistic bacteria-killing combination to kill bacteria or treat infection.

### FIELD OF THE INVENTION

The invention is directed to a microorganism-killing combination comprising a photosensitizer and a chitosan. In particular, the chitosan is in an amount effective to enhance the microorganism-killing effect in a photodynamic process.

### BACKGROUND OF THE INVENTION

The emergence of antibiotic resistance amongst pathogenic bacteria has led to a major research effort to find alternative antibacterial therapies and treatments. A new promising approach to the treatment of bacterial infections is called bacterial photodynamic inactivation (PDI) or photodynamic therapy (PDT), a process in which cells are treated with an agent that makes them susceptible to death by exposure to light. These agents, called photosensitizers, are generally macrocyclic compounds that exhibit no or minimal inherent toxicity but result in the generation of cytotoxic reactive oxygen species when excitation occurs with light of an appropriate wavelength. The administration of a photosensitizer is preferentially accumulated in the microbial cells. The subsequent irradiation with visible light, in the presence of oxygen, specifically produces cell damage that inactivates the microorganisms. (Durantini, Edgardo N., Current Bioactive Compounds Volume 2, Number 2, June 2006, pp. 127-142(16)). A photosensitizer is a chemical compound that can be excited by light of a specific wavelength. This excitation uses visible or near-infrared light. Usually, the photosensitizer is excited from a ground singlet state to an excited singlet state. When the photosensitizer and an oxygen molecule are in proximity, an energy transfer can take place that allows the photosensitizer to relax to its ground singlet state, and create an excited singlet state in the oxygen molecule. Singlet oxygen is a very aggressive chemical species and will very rapidly react with any nearby biomolecules. Bacteria and viral organisms are extremely sensitive to reactive oxygen species such as singlet oxygen.

U.S. Pat. No. 5,798,238 discloses that viral and bacterial contaminants present in biological solutions are inactivated by mixing one of a class of photosensitizer with said solution and irradiating the mixture. U.S. Pat. No. 6,469,052 provides new psoralens and methods of synthesis of new psoralens having an enhanced ability to inactivate pathogens in the presence of ultraviolet light. The new psoralens are effective against a wide variety of pathogens.

Chitosan is the deacetylated derivative of the polysaccharide chitin [B-(1-4)-poly-N-acetyl-D-glucosamine], an abundant natural by-product of the crab and shrimp industries. Chitosan has been used and/or suggested for use for a wide variety of purposes, including, by way of illustration, flocculation of bacteria, yeasts and microfungi from suspensions containing the same; flocculation of industrial wastes such as proteins in liquid wastes from packing houses, poultry, fish and vegetable processing plants, whey, leather tanning wastes, Kraft paper mill wastes and suspended solids from mine tailings; preparation of membranes which have ion exchange properties and have various permeability properties in regard to moisture and gases such as oxygen, nitrogen and carbon dioxide; chelation and column chromatography metal, enzyme and virus separation procedures; as viscosity builders for various foods, cosmetics, drugs, etc.; broad-spectrum antifungal uses such as preventing the growth of pathogenic fungi which normally infect peas and other plant products; inhibition of the fermentation of yeasts in various food products; and application to seeds prior to planting to prevent fungal diseases. U.S. Pat. No. 4,957,908 discloses pyrithione salt, namely chitosan pyrithione, which is characterized by a combination of a slow release of pyrithione and excellent antimicrobial activity. Chung Y C et al. indicates that the antibacterial activity of chitosan and the surface characteristics of the cell wall are closely related (Acta Pharmacol Sin 2004 July; 25 (7): 932-936). It has been reported that the antibacterial activity of chitosan is influenced by its molecular weight, degree of deacetylation, concentration in solution, and pH of the medium (Xiao Fei Liu et al., J Appl Polym Sci 79: 1324-1335, 2001). Raymond Bonnett et al. uses photosensitizers incorporated into a chitosan membrane to evaluate photomicrobicidal activity in static systems against Escherichia coli and shows that the ZnPcS/chitosan membrane as the photosensitizing surface is a significant photokill of E. coli (Water Research 40 (2006), 1269-1275). This prior art reference employs chitosan as a biopolymer to facilitate contact between the photosensitizer and the microorganism, whereas it does not teach or suggest that chitosan provides any advantageous effect in killing and/or inhibiting microorganisms.

However, infectious diseases obviously remain an unsolved problem, due largely to the emergence of multiple antibiotic resistant strains of bacteria, newly discovered viral diseases, and the spread of fungal and protozoan diseases. Although the photodynamic effect has been used against microbial infection, there is still a need for an agent and process to improve the ability of the currently known photodynamic agents.

### SUMMARY OF THE INVENTION

The invention relates to a microorganism-killing combination comprising a photosensitizer in an amount effective to kill microorganisms in a photodynamic process and a chitosan in an amount effective to enhance the microorganism-killing effect of the photosensitizer in the photodynamic process.

The invention also relates to a method of killing microorganisms in a subject which comprises the steps of administering the combination of the invention to the subject and irradiating the photosensitizer in the combination, thereby killing the microorganisms.

The invention also relates to a method of treating microorganism infection in a subject which comprises the steps of administrating the combination of the invention to the subject and irradiating the photosensitizer in the combination, thereby treating the microorganism infection.

### BRIEF DESCRIPTION OF THE DRAWINGS

FIG. 1 shows the effect of chitosan on Hp mediated photodynamic inactivation (PDI) against Staphylococcus aureus (BCRC 10780).

FIG. 2 shows the effect of chitosan on photodynamic inactivation (PDI) against different strains of Methicillin-resistant Staphylococcus aureus (MRSA) (ATCC 49476 & MRSA ATCC 33592). FIG. 2A is the killing effect on MRSA strain (ATCC 49476) and FIG. 2B is the killing effect on MRSA strain (ATCC 33592).

FIG. 3 shows the effect of chitosan on photodynamic inactivation (PDI) against other gram positive bacteria, Streptococcus epidermidis (ATCC 12228) (FIG. 3A) and Streptococcus pyogenes (ATCC 19615) (FIG. 3B).

FIG. 4 shows the effect of chitosan on Hp mediated photodynamic inactivation (PDI) against Pseudomonas aeruginosa.

FIG. 5 shows the survival fraction of Staphylococcus aureus (BCRC 10780) treated with Rose Bengal (FIG. 5A), Methylene Blue (FIG. 5B) and Chlorin e6 (FIG. 5C) mediated photodynamic inactivation with or without chitosan.

FIG. 6 shows the survival fraction of Candida albicans treated with Toluidine blue (FIG. 6A) or Methylene Blue (FIG. 6B) mediated photodynamic inactivation with or without 0.6% chitosan.

FIG. 7 shows the effect of various molecular weight of chitosan on Hp mediated photodynamic inactivation (PDI) against Staphylococcus aureus.

### DETAILED DESCRIPTION OF THE INVENTION

The invention has unexpectedly discovered that chitosan can enhance the microorganism-killing efficacy of photosensitizers. It is known in the art that chitosan can inhibit growth of microorganisms but cannot kill microorganisms. Surprisingly, the invention has found that chitosan enhances the efficacy of PDI or PDT and can kill microorganisms. In particular, a very low level of chitosan is sufficient to enhance the microorganism-killing effect as stated above.

The invention provides a microorganism-killing combination comprising a photosensitizer in an amount effective to kill microorganisms in a photodynamic process and a chitosan in an amount effective to enhance the microorganism-killing effect of the photosensitizer in the photodynamic process.

The invention also provides a method of killing microorganisms in a subject which comprises the steps of administering the combination of the invention to the subject and irradiating the photosensitizer in the combination, thereby killing the microorganisms.

The invention also provides a method of treating microorganism infection in a subject which comprises the steps of administering the combination of the invention to the subject and irradiating the photosensitizer in the combination, thereby treating the microorganism infection.

According to the embodiments of the invention, the photosensitizer and the chitosan in the combinations and methods of the invention can be administered concomitantly (e.g., as an admixture), separately but simultaneously (e.g., via separate usage routes into the same subject), or sequentially (e.g., one of the compounds or agents is given first followed by the second). Thus, the term “combination” or “combined” is used to refer to concomitant, simultaneous, or sequential administration of two or more agents.

According to one embodiment of the invention, the combination of the invention can achieve a synergistic effect in killing bacteria, so it also is a synergistic combination. The phrase “synergistic combination” according to the invention is a new combination and has the activity superior to that of the single components thereof.

The phrase “amount effective to kill microorganisms in a photodynamic process” according to the invention is the amount of the photosensitizer used in the combination of the invention to kill the microorganisms through a photodynamic process. According to the invention, the amount of photosensitizer used in the inventions is at least 0.01 μM. Preferably, the amount of photosensitizer is 0.01-0.25 μM, 0.01-0.5 μM, 0.01 μM to 1 mM, 0.01 μM to 1.25 mM or 0.01 μM to 1.5 mM. More preferably, the amount of photosensitizer is 0.01-0.25 μM, 0.25-0.5 μM, 0.5 μM to 0.5 mM, 0.5 mM to 1 mM or 1 mM to 1.25 mM.

The phrase “amount effective to enhance the microorganism-killing effect of the photodynamic process” according to the invention is the amount of the chitosan used in the combination of the invention to enhance the microorganism-killing effect of the photosensitizer in a photodynamic process. The amount of chitosan is at least 0.001% (w/v). Preferably, the amount of chitosan is 0.005%-0.025% (w/v), 0.005%-0.01% (w/v), 0.005%-0.25% (w/v), 0.005%-0.6% (w/v), 0.005%-1% (w/v) or 0.005%-5% (w/v). More preferably, the amount of chitosan is 0.025%-0.01% (w/v), 0.01%-0.25% (w/v), 0.25%-0.6% (w/v), 0.6%-1% (w/v) or 1%-5% (w/v). The amounts of photosensitizer and chitosan, however, may vary depending upon the microorganism to be treated. They can be determined by routine experimentation and is within the judgement of those of skill in the art.

The phrase “photodynamic process” according to the invention refers to photodynamic inactivation (PDI) or photodynamic therapy (PDT), which is a process comprising a photo-reactive agent that resides in close proximity to the target cell, photosensitizing light, and oxygen that comes into an interactive area.

Chitosan can be created by N-deacetylation of the chitin polymer. It is a linear polysaccharide composed of randomly distributed β-(1-4)-linked D-glucosamine (deacetylated unit) and N-acetyl-D-glucosamine (acetylated unit). Chitosan exhibits antimicrobial activity against some strains of filamentous fungi, yeasts, and bacteria; however, it does not have activity or only has poor activity in killing microorganisms. As used herein, the chitosan used in the combination of the invention refers to a native chitosan or its derivatives. Any commercially available chitosan can be used in the invention. Preferably, the molecular weight of chitosan that is used to combine PDI to enhance the bacterial killing ranges from 0.5 kDa to 1000 kDa.

As used herein, a photosensitizer refers to a substance which, upon irradiation with electromagnetic energy of the appropriate wavelength, usually light of the appropriate wavelength, produces a cytotoxic effect. The invention may be practiced with a variety of synthetic and naturally occurring photosensitizers. Many photosensitizers produce singlet oxygen. Upon electromagnetic irradiation at the proper energy level and wavelength, such a photosensitizer molecule is converted to an energized form. Singlet oxygen is highly reactive, and is toxic to a proximal target organism. Photosensitizers include, but are not limited to, hematoporphyrins, such as hematoporphyrin HCl and hematoporphyrin esters; dihematophorphyrin ester; hematoporphyrin IX and its derivatives; 3,1-meso tetrakis (o-propionamidophenyl) porphyrin; hydroporphyrins such as chlorin, herein, and bacteriochlorin of the tetra (hydroxyphenyl) porphyrin series, and synthetic diporphyrins and dichlorins; o-substituted tetraphenyl porphyrins (picket fence porphyrins); chlorin e6; monoethylendiamine monamide; mono-1-aspartyl derivative of chlorin e6, and mono- and diaspartyl derivatives of chlorin e6; the hematoporphyrin mixture Photofrin II; benzophorphyrin derivatives (BPD), including benzoporphyrin monoacid Ring A (BPD-MA), tetracyanoethylene adducts, dimethyl acetylene dicarboxylate adducts, Diels-Adler adducts, and monoacid ring “a” derivatives; a naphthalocyanine; toluidine blue O; aluminum sulfonated and disulfonated phthalocyanine ibid.; and phthalocyanines without metal substituents, and with varying other substituents; a tetrasulfated derivative; sulfonated aluminum naphthalocyanines; methylene blue; nile blue; crystal violet; azure β chloride; toluidine blue; and rose bengal. The photosensitizer used in the invention is preferably hematoporphyrin, chlorine e6, toluidine blue, Rose Bengal, or methylene blue.

Other potential photosensitizers include, but are not limited to, pheophorbides such as pyropheophorbide compounds, anthracenediones; anthrapyrazoles; aminoanthraquinone; phenoxazine dyes; phenothiazine derivatives; chalcogenapyrylium dyes including cationic selena- and tellura-pyrylium derivatives; verdins; purpurins including tin and zinc derivatives of octaethylpurpurin and etiopurpurin; benzonaphthoporphyrazines; cationic imminium salts; and tetracyclines.

According to the invention, the photosensitizer used in the combination of the invention can be in any form, preferably free form, micellar form, liposomal form or other carriers. The photosensitizer is more preferably in micellar or liposomal form.

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium. Liposomes can range in size from several nanometers to several micrometers in diameter. Liposome morphological types are broadly categorized as either multilamellar, unilamellar or micellar. A liposome used according to the invention can be made by different methods, as would be known to one of ordinary skill in the art. Illustrative examples of phospholipids for the preparation of liposome include lecithin, sphingomyelin, dipalmitoylphosphatidylcholine, etc. Representative steroids include cholesterol, chlorestanol, lanosterol, and the like. Representative charge amphiphilic compounds generally contain 12 to 30 carbon atoms. Mono- or dialkyl phosphate esters, or alkylamines, e.g. dicetyl phosphate, stearyl amine, hexadecyl amine, dilaurylphosphate, and the like are representative. The liposome sacs can be prepared by vigorous agitation in the solution. Further details with respect to the preparation of liposomes are set forth in U.S. Pat. No. 4,342,826 and PCT International Publication No. WO 80/01515, both of which are incorporated by reference. A micelle is defined as a water soluble or colloidal structure or aggregate (also called a nanosphere or nanoparticle) composed of one or more amphiphilic molecules. Micelles range in size from 5 to about 2000 nanometers, preferably from 10 to 400 nm Amphiphilic molecules are those that contain at least one hydrophilic (polar) moiety and at least one hydrophobic (nonpolar) moiety. The micelles can be composed of either a single monomolecular polymer containing hydrophobic and hydrophilic moieties or an aggregate mixture containing many amphiphilic (i.e. surfactant) molecules formed at or above the critical micelle concentration (CMC), in a polar (i.e. aqueous) solution. The micelle is self-assembled from one or more amphiphilic molecules where the moieties are oriented to provide a primarily hydrophobic interior core and a primarily hydrophilic exterior.

The effect of the combination of the invention is triggered by photodynamic inactivation of bacteria or the effect of chitosan, depending on the sequence of administration of the photosensitizer and chitosan. In use, the photosensitizers of the invention are illuminated/irradiated, i.e. activated, using conventional techniques known in the field of photodynamic inactivation. Preferably, the photosensitizers are illuminated/irradiated at a wavelength between 400 nm and 800 nm More preferably, the photosensitizers are illuminated/irradiated at a wavelength corresponding to one or more of the absorption windows for porphyrin, which lie at around 417 nm (Soret band), 485 nm, 515 nm, 550 nm, 590 nm and 650 nm Illumination/irradiation of the appropriate wavelength for a given compound can be administered by a variety of methods. These methods include but are not limited to the administration of a laser, a nonlaser, or broadband light. Irradiation can be produced by extracorporeal or intraarticular generation of light of the appropriate wavelength.

According to the invention, the combination of the invention can be further formulated with pharmaceutically acceptable excipients. The excipients described herein, for example, vehicles, adjuvants, carriers or diluents, are well known to those who are skilled in the art and are readily available to the public. It is preferred that the pharmaceutically acceptable carrier be one which is chemically inert to the combination of the invention and one which has no detrimental side effects or toxicity under the conditions of use.

For injectable formulations, the requirements for effective pharmaceutical carriers of injectable compositions are well known to those of ordinary skill in the art. It is preferred that such injectable compositions be administered intramuscularly, intravenously, or intraperitoneally.

Topical formulations are well known to those of skill in the art and are suitable for application to the skin. The use of patches and ointments is also within the limits of the skill in the art.

Formulations suitable for oral administration can consist of liquid solutions, capsules, sachets, tablets, lozenges, and troches, powders, suspensions and suitable emulsions.

The combinations of the invention are generally widely effective against microorganisms, preferably bacteria and fungi, more preferably bacteria, even more preferably gram positive or negative bacteria. More preferably, the combinations of the invention are able to kill Staphylococcus, methicillin-resistant Staphylococcus aureus, Streptococcus, Pseudomonas Staphylococcus or Candida. Most preferably, the combinations of the invention are able to kill Staphylococcus aureus, methicillin-resistant Staphylococcus aureus, Streptococcus epidermidis, Streptococcus pyogenes, Pseudomonas aeruginosa, Staphylococcus aureus or Candida albicans.

It will now be apparent to those skilled in the art that other embodiments, improvements, details, and uses can be made that are consistent with the letter and spirit of the foregoing disclosure and within the scope of this patent and the appended claims.

### EXAMPLE

### Example 1

### Effect of Chitosan on Hp Mediated Photodynamic Inactivation (PDI) Against Staphylococcus aureus (BCRC 10780)

The micelle Hp was prepared on the basis of the thin film formulation method (Sezgin, Z. N. et al., Int J Pharm 332:161-7). Hp and Pluronic®F127/Synperonic®L122 at a ratio of 1:4 (v/v) were added to a flask and mixed. The resulting solution was concentrated under a reduced pressure at 25° C. (Pluronic®F127) or 37° C. (Synperonic®L122) for 20 minutes so that the solvent was removed and a thin film was formed on the bottom of the flask. 1 ml of ddH₂O was added to form 10% w/v of micelle solution and then the resulting solution was shaken in a 25° C. water bath with a sonicator for 20 minutes. The solution was deposited at room temperature overnight and then filtered with 0.22 μm PVDF filter to remove free-form Hp. The encapsulated Hp with micelle was called micelle Hp.

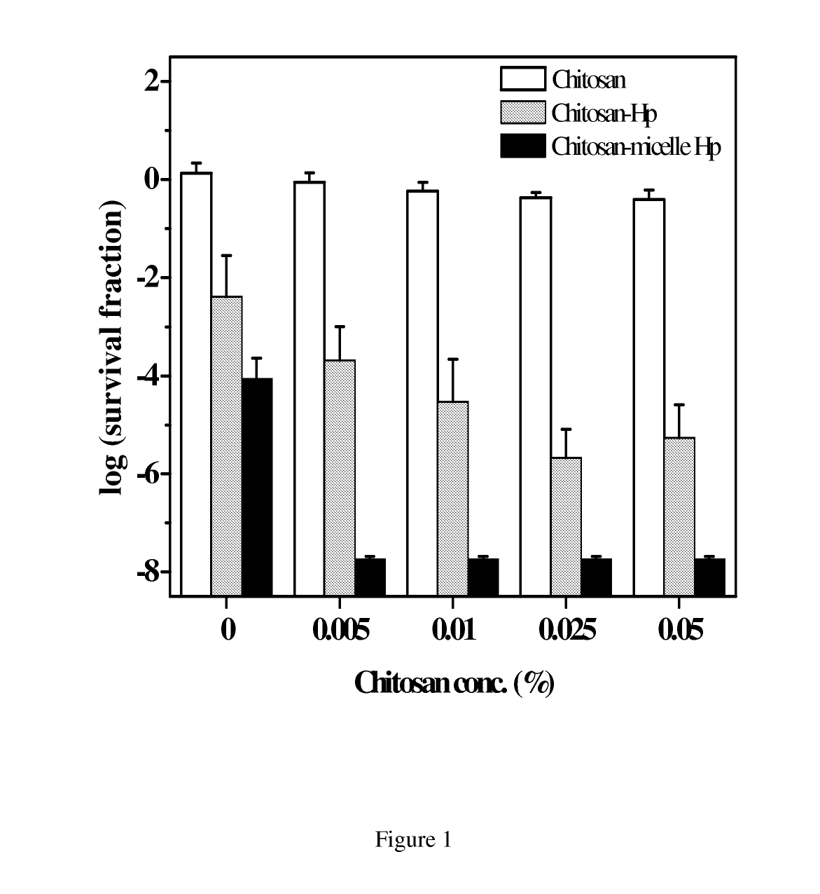

0.1 ml of a cell suspension of Staphylococcus aureus containing approximately 10⁸ CFU per ml was transferred into a well. Then, 0.1 ml of the free-form Hp or micelle Hp solution (0.1 μM) containing different concentrations of chitosan was added to the well and incubated in the dark for 30 min, and then subjected to illumination using 630±5 nm LED light source for 25 J/cm². Irradiated as well as non-irradiated bacterial cells were serially diluted 10-fold with PBS and the colonies formed after 18 hours of incubation at 37° C. were counted. Plate count was performed with the standard method. Briefly, aliquots (10 μl) of appropriate dilutions (from 10⁻¹ to 10⁻⁵) of each sample were placed on TSB agar plates and incubated at 37° C. in darkness for 18 hours. The survival fraction was calculated as N_{PDI}/N₀, where N_{PDI} is the number of CFU per ml after photodynamic inactivation and N₀ is the number of CFU per ml in the initial sample. As shown in FIG. 1, the killing effect of Hp-PDI was greatly enhanced in the presence of chitosan at 0.005% (w/v). In particular, the bacteria were almost all killed in the micelle Hp group with 0.005% (w/v) chitosan. Further increasing the chitosan concentration to 0.05% (w/v) seemed to increase the killing efficiency of the Hp free-form group but not the micelle group since the bacteria in the latter were totally eradicated in the presence of 0.005% (w/v) chitosan.

### Example 2

### Effect of Chitosan on Photodynamic Inactivation (PDI) Against Different Strains of Methicillin-Resistant Staphylococcus aureus (MRSA) (ATCC 49476 & MRSA ATCC 33592)

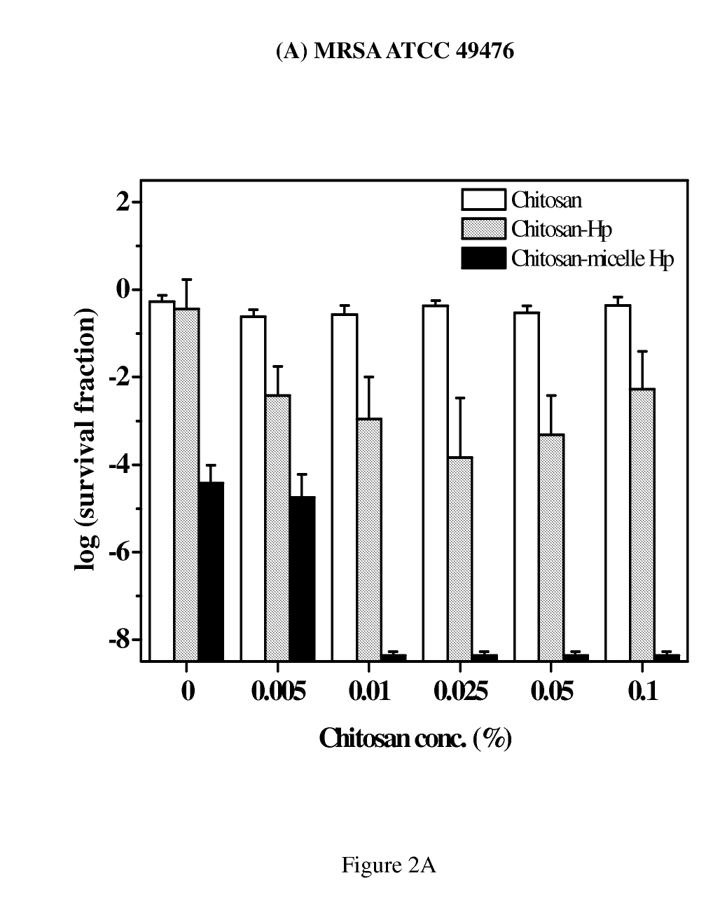

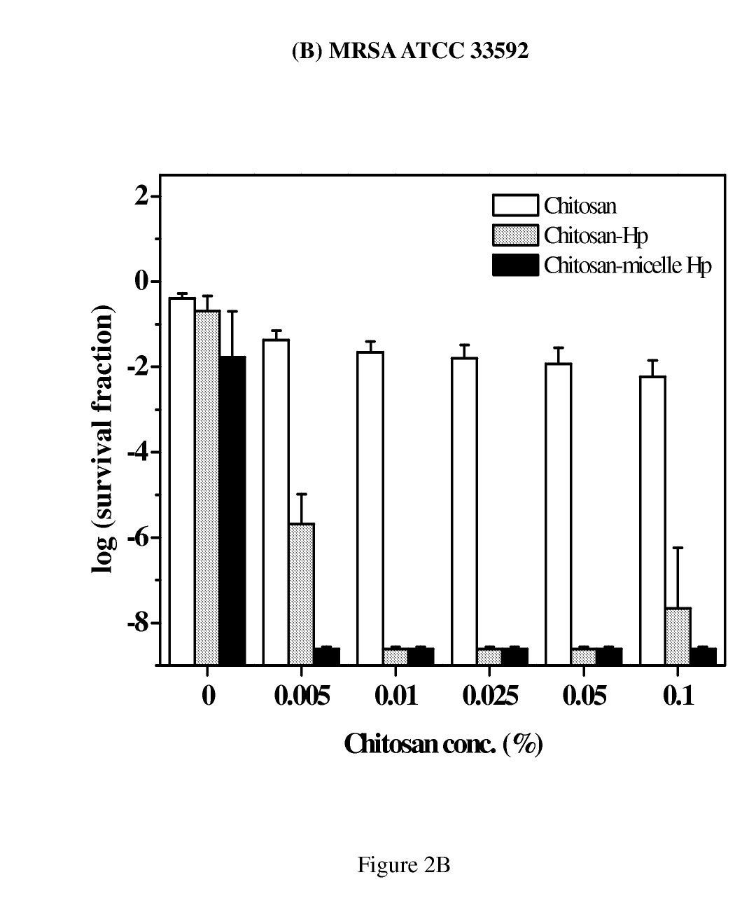

The preparation of micelle Hp is as stated in Example 1. 0.1 ml of a cell suspension of the MRSA bacteria containing approximately 10⁸ CFU per ml was transferred into a well. Then, 0.1 ml of the free-form Hp or micelle Hp solution (0.1 μM or 0.25 μM) containing different concentrations of chitosan was added to the well and incubated in the dark for 30 min unless otherwise specified, and then subjected to illumination using 630±5 nm LED light source for 25 J/cm². Irradiated as well as non-irradiated bacterial cells were serially diluted 10-fold with PBS and the colonies formed after 18 h of incubation at 37° C. were counted. Plate count was performed with the standard method. Briefly, aliquots (10 μl) of appropriate dilutions (from 10⁻¹ to 10⁻⁵) of each sample were placed on TSB agar plates and incubated at 37° C. in darkness for 18 hours. The survival fraction was calculated as N_{PDI}/N₀, where N_{PDI} is the number of CFU per ml after photodynamic inactivation and N₀ is the number of CFU per ml in the initial sample. As shown in FIG. 2A, it is obvious that using 0.25 μM Hp to perform PDI against one of the MRSA strain (ATCC 49476) resulted in a complete eradication of the bacteria in the presence of 0.01% (w/v) chitosan. As shown in FIG. 2B, MRSA strain (ATCC 33592) seemed to be much susceptible to 0.1 μM Hp-PDI; the presence of chitosan enhanced the PDI effect and resulted in a complete eradication of the microbe at a very low concentration: 0.005% (w/v) when micelle Hp was used, and 0.01% (w/v) when free-form Hp was used.

### Example 3

### Effect of Chitosan on Photodynamic Inactivation (PDI) Against Streptococcus epidermidis (ATCC 12228) and Streptococcus pyogenes (ATCC 19615)

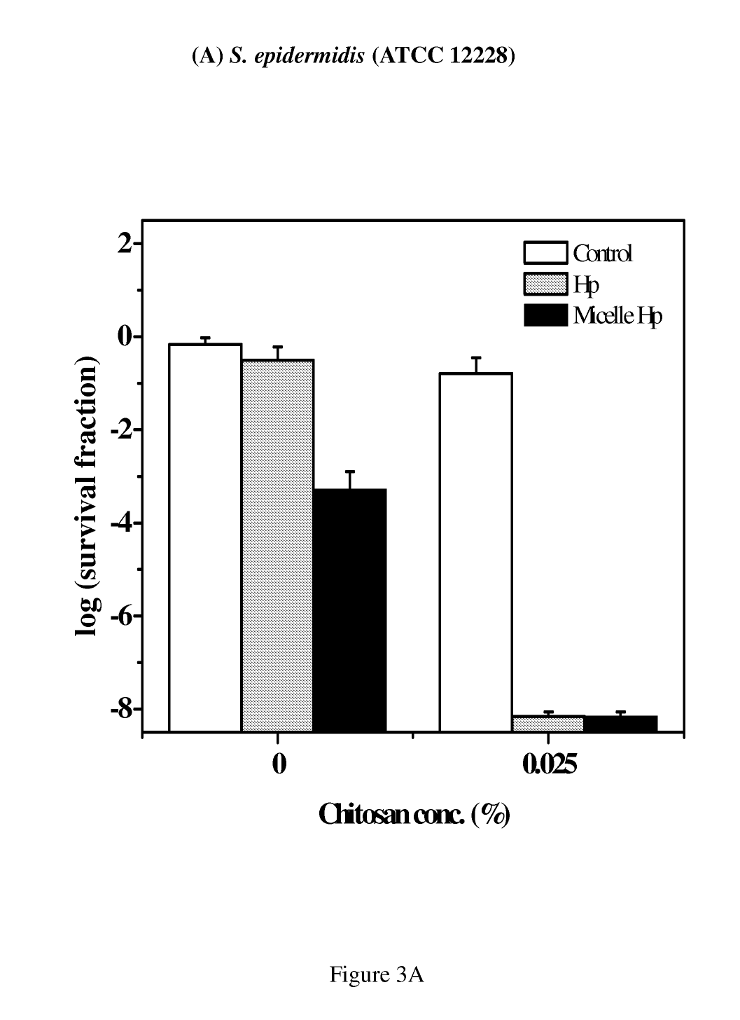

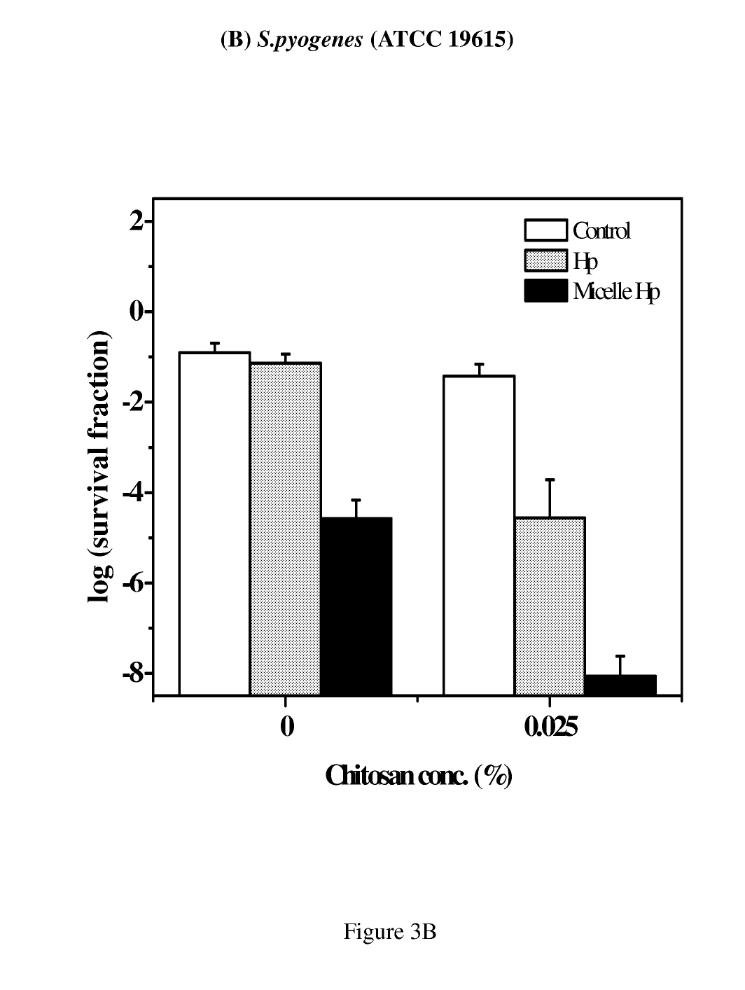

The preparation of micelle Hp is as stated in Example 1. 0.1 ml of a cell suspension of S. epidermidis (A) or S. pyogenes (B) containing approximately 10⁸ CFU per ml was transferred into a well. Then, 0.1 ml of free-form or micelle Hp solution (0.1 μM) in the presence or absence of 0.025% (w/v) chitosan was added to the well and incubated in the dark for 30 min, and then subjected to illumination using 630±5 nm LED light source for 25 J/cm². Irradiated as well as non-irradiated bacterial cells were serially diluted 10-fold with PBS and the colonies formed after 18 h of incubation at 37° C. were counted. Plate count was performed with the standard method. Briefly, aliquots (10 μl) of appropriate dilutions (from 10⁻¹ to 10⁻⁵) of each sample were placed on TSB agar plates and incubated at 37° C. in darkness for 18 hours. The survival fraction was calculated as N_{PDI}/N₀, where N_{PDI} is the number of CFU per ml after photodynamic inactivation and N₀ is the number of CFU per ml in the initial sample. The results are as shown in FIG. 3. Micelle Hp was able to produce a stronger PDI effect compared to the free form group, and the presence of chitosan at 0.025% (w/v) further assisted micelle Hp, resulting in a complete eradication of the bacteria. PDI of Staphylococcus epidermidis (ATCC 12228) using the free-form Hp also resulted in a complete killing through the aid of chitosan, and without the chitosan, the PDI effect was not obvious (FIG. 3A). Streptococcus pyogenes (ATCC 19615) using the free-form Hp also resulted in a significant decrease in bacteria number through the aid of chitosan, but was not able to induce a complete eradication (FIG. 3B).

### Example 4

### Effect of Chitosan on Hp Mediated Photodynamic Inactivation (PDI) Against Pseudomonas aeruginosa

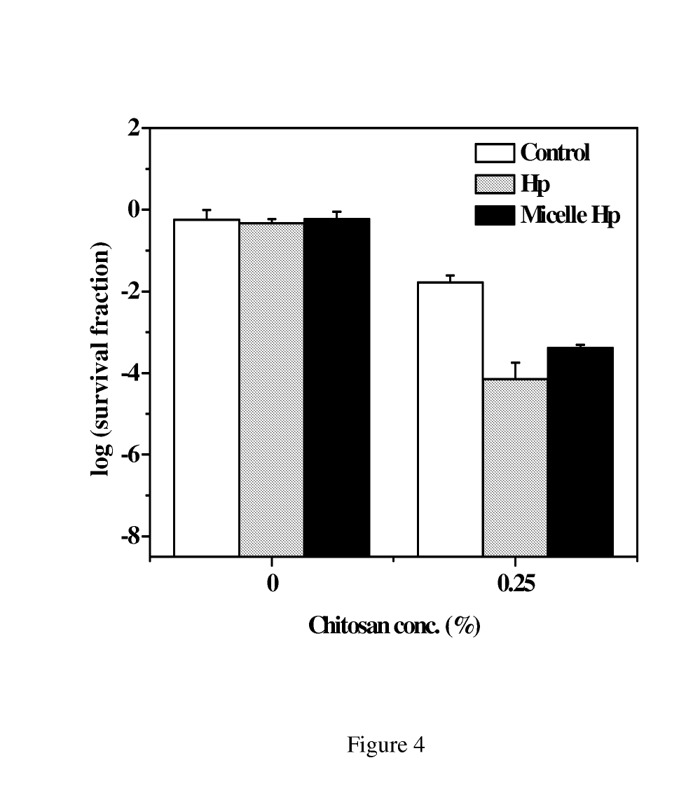

The preparation of micelle Hp is as stated in Example 1. 0.1 ml of a cell suspension of Pseudomonas aeruginosa containing approximately 10⁸ CFU per ml was transferred into a well. Then, 0.1 ml of the free-form or micelle Hp solution (25 μM) in the presence or absence of 0.25% chitosan was added to the well and incubated in the dark for 2 hours, and then subjected to illumination using 630±5 nm LED light source for 50 J/cm². Irradiated as well as non-irradiated bacterial cells were serially diluted 10-fold with PBS and the colonies formed after 18 hours of incubation at 37° C. were counted. Plate count was performed with the standard method. Briefly, aliquots (10 μl) of appropriate dilutions (from 10⁻¹ to 10⁻⁵) of each sample were placed on TSB agar plates and incubated at 37° C. in darkness for 18 h. The survival fraction was calculated as N_{PDI}/N₀, where N_{PDI} is the number of CFU per ml after photodynamic inactivation and N₀ is the number of CFU per ml in the initial sample. The results are as shown in FIG. 4. PDI has been reported to be more effective against the gram-positive bacteria and less effective against the gram-negative strains. As can be seen from the results shown in FIG. 4, Hp-PDI was totally not effective against Pseudomonas aeruginosa even with 25 μM Hp. However, the presence of chitosan could enhance the effect of the PDI using the free-form Hp or micelle Hp against this gram-negative bacterial strain. In the presence of 0.25% (w/v) chitosan, either free-form Hp or micelle Hp has advantageous effect in increasing the killing of gram-negative Pseudomonas aeruginosa.

### Example 5

### Survival Fraction of Staphylococcus aureus (BCRC 10780) Treated with Rose Bengal (A), Methylene Blue (B) and Chlorin e6 (C) Mediated Photodynamic Inactivation with or without Chitosan

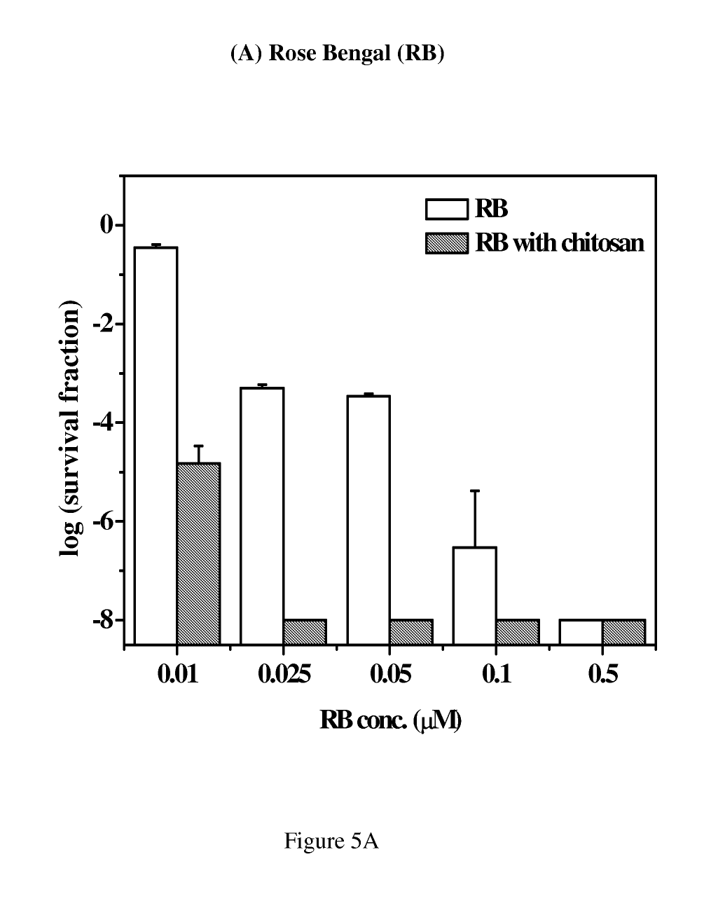

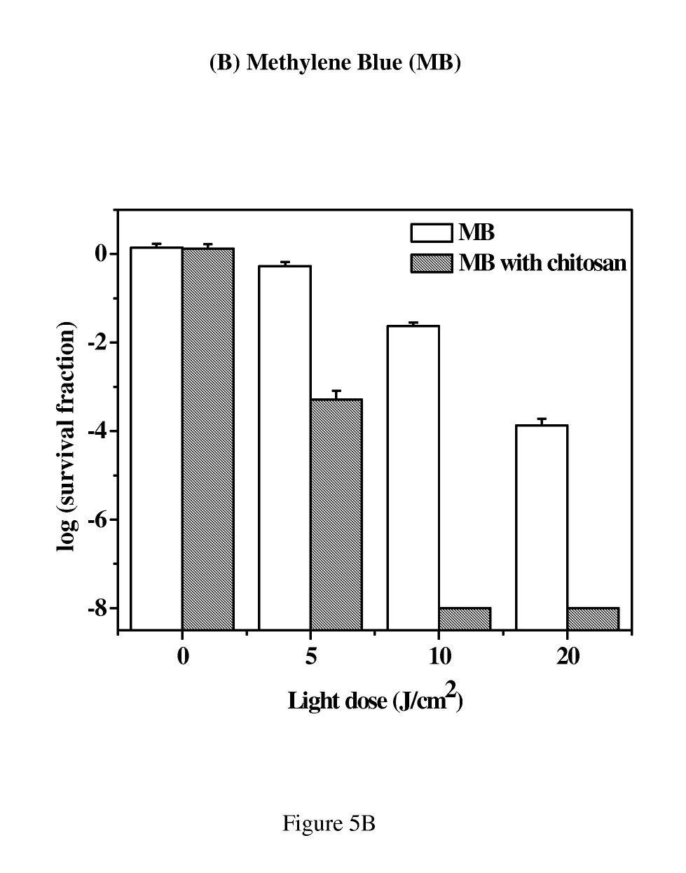

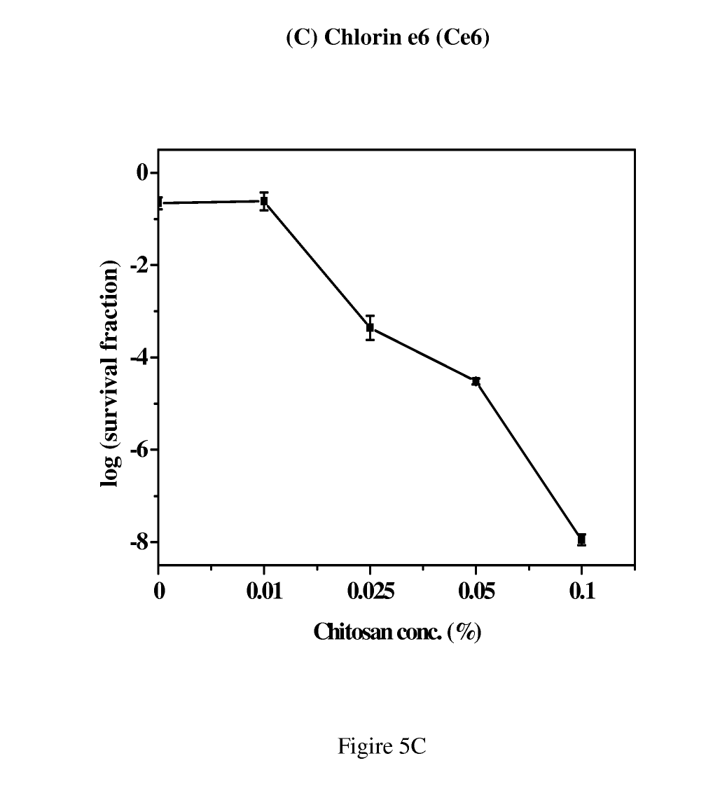

The preparation of micelle Hp is as stated in Example 1. 0.1 ml of a cell suspension of Staphylococcus aureus containing approximately 10⁸ CFU per ml was transferred into a well. In the PDI treatment using Rose Bengal (RB), 0.1 ml of different concentrations of RB in the presence or absence of 0.025% (w/v) chitosan was added to the well and incubated in the dark for 30 minutes and then subjected to illumination using 630±5 nm LED light source for 25 J/cm². In the PDI treatment using methylene blue (MB), 0.1 ml of 2 μM MB solution in the presence or absence of 0.025% (w/v) chitosan was added to the well and incubated in the dark for 30 minutes and then subjected to illumination using 650±5 nm LED light source for different light doses (0, 5, 10, 20 J/cm²). In the PDI treatment using chlorine e6 (Ce6), 0.1 ml of 0.05 μM Ce6 solution in the presence or absence of chitosan (0, 0.01%, 0.025%, 0.05%, 0.1% (w/v)) was added to the well and incubated in the dark for 30 minutes and then subjected to illumination using 650±5 nm LED light source for 20 J/cm². Irradiated as well as non-irradiated bacterial cells were serially diluted 10-fold with PBS and the colonies formed after 18 hours of incubation at 37° C. were counted. Plate count was performed with the standard method. Briefly, aliquots (10 μl) of appropriate dilutions (from 10⁻¹ to 10⁻⁵) of each sample were placed on TSB agar plates and incubated at 37° C. in darkness for 18 hours. The survival fraction was calculated as N_{PDI}/N₀, where N_{PDI} is the number of CFU per ml after photodynamic inactivation and N₀ is the number of CFU per ml in the initial sample. The results are as shown in FIG. 5. The results showed that using 0.025 μM RB and 25 J/cm² to perform the PDI was not able to result in complete eradication, and complete removal of the bacteria could be achieved through the aid of 0.025% chiotosan in the system. Using 2 μM MB to perform the PDI was found to be dependent on light dose: 0 J/cm² was ineffective, 5 J/cm² was slightly effective, and 10-20 J/cm² was apparently more effective than the lower dose. In the presence of 0.025% (w/v) chitosan, the PDI effect was significantly enhanced, and 10-20 J/cm² could result in complete eradication. At the concentration of 0.05 μM Ce6, chitosan can significantly enhance the PDI bacterial killing effect and was shown in a concentration dependent manner (0.01-0.1 μM). In the presence of 0.1% (w/v) chitosan, the PDI effect (10 J/cm²) could result in complete eradication.

### Example 6

### Survival Fraction of Candida albicans Treated with Toluidine Blue (A) or Methylene Blue (B) Mediated Photodynamic Inactivation with or without 0.6% Chitosan

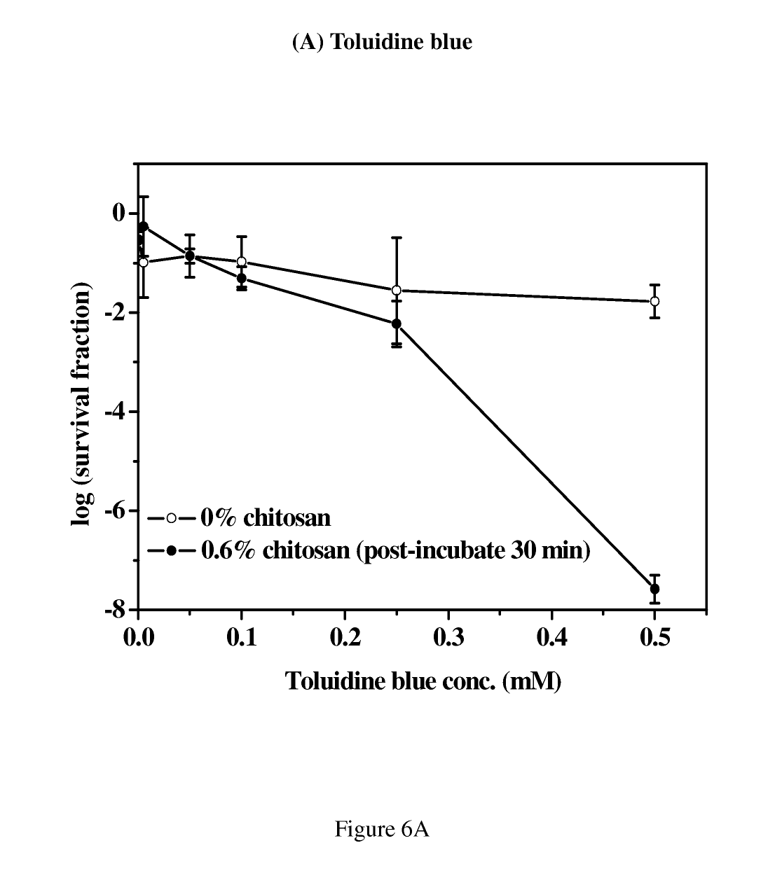

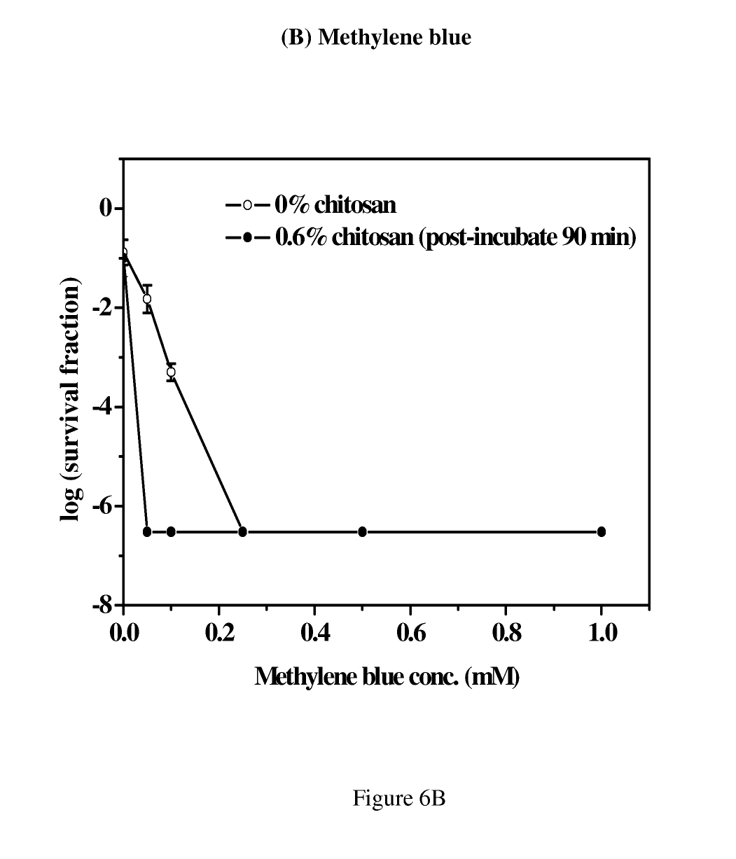

0.1 ml of a cell suspension of Candida albicans containing approximately 10⁸ CFU per ml was transferred into the well of a 96-well plate. In the PDI treatment using toluidine blue (TB), 0.1 ml of different concentrations of TB was added to the well and incubated in the dark for 30 minutes and then subjected to illumination using 630±5 nm LED light source for 50 J/cm². After light irradiation, 0.6% chitosan was added and further incubated for 30 minutes before plate count. In the PDI treatment using MB, 0.1 ml of different concentrations of MB was added to the well and incubated in the dark for 30 minutes and then subjected to illumination using 630±5 nm (toluidine) or 650±5 nm (methylene blue) LED light source for 50 J/cm². After light irradiation, 0.6% (w/v) chitosan was added and further incubated for 30 minutes (toluidine) or 90 minutes (methylene blue) before plate count. Irradiated as well as non-irradiated bacterial cells were serially diluted 10-fold with PBS and the colonies formed after 18 hours of incubation at 37° C. were counted. Plate count was performed with the standard method. Briefly, aliquots (10 μl) of appropriate dilutions (from 10⁻¹ to 10⁻⁵) of each sample were placed on SAB agar plates and incubated at 37° C. in darkness for 18 hours. The survival fraction was calculated as N_{PDI}/N₀, where N_{PDI} is the number of CFU per ml after photodynamic inactivation and No is the number of CFU per ml in the initial sample. The results are as shown in FIG. 6. The survival fraction of Candida albicans was found to be greatly reduced when 0.6% (w/v) chitosan was added after the PDI using TB. 0.5 mM TB combined with light illumination using 630±5 nm LED light source for 50 J/cm² was partially effective without chitosan, and adding the 0.6% (w/v) chitosan 30 minutes after the PDI resulted in complete eradiation of the fungi. The survival fraction of Candida albicans was found to be greatly reduced when 0.6% (w/v) chitosan was added after the PDI using MB. 0.5 mM MB combined with light illumination using 630±5 nm LED light source for 50 J/cm² was partially effective without chitosan, and adding the 0.6% (w/v) chitosan 90 minutes after the PDI resulted in complete eradiation of the fungi.

### Example 7

### Effect of Various Molecular Weight of Chitosan on Hp Mediated Photodynamic Inactivation (PDI) Against Staphylococcus aureus (BCRC 10780)

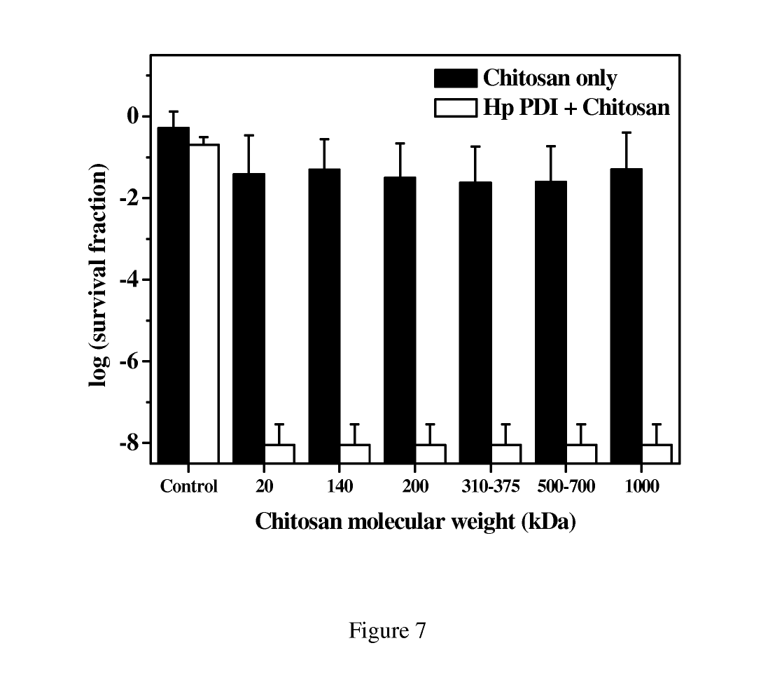

0.1 ml of a cell suspension of Staphylococcus aureus containing approximately 10⁸ CFU per ml was transferred into a well. Then, 0.1 ml of the free-form Hp solution (0.1 μM) was added to the well and incubated in the dark for 30 minutes. After subjected to illumination using 653±5 nm LED light source for 25 J/cm². Then, 0.1% (w/v) chitosan with different molecular weight was added and further incubated for 30 minutes before plate count. Plate count was performed with the standard method. Briefly, aliquots (10 μl) of appropriate dilutions (from 10⁻¹ to 10⁻⁵) of each sample were placed on TSB agar plates and incubated at 37° C. in darkness for 18 hours. The survival fraction was calculated as N_{PDI}/N₀, where N_{PDI} is the number of CFU per ml after photodynamic inactivation and N₀ is the number of CFU per ml in the initial sample. As shown in FIG. 7, chitosan can enhance the Hp-PDI killing. In particular, the bacteria were all killed when combining the PDI with 0.15% chitosan with 20, 140, 200, 310˜375, 500˜700 and 1000 kDa. The detailed results are shown in FIG. 7.

What is claimed is:

1. A microorganism-killing combination comprising a photosensitizer in an amount effective to kill microorganisms in a photodynamic process and a chitosan in an amount effective to enhance the microorganism-killing effect of the photosensitizer in the photodynamic process.

2. The combination of claim 1, wherein the photosensitizer and the chitosan are administered concomitantly, separately but simultaneously, or sequentially.

3. The combination of claim 1, wherein the photosensitizer and the chitosan are administrated concomitantly

4. The combination of claim 1, wherein the microorganism is bacteria or fungi.

5. The combination of claim 4, wherein the bacteria is gram-positive or gram-negative bacteria.

6. The combination of claim 5, wherein the gram-positive bacteria is Staphylococcus, methicillin-resistant Staphylococcus, or Streptococcus, and the gram-negative bacteria is Pseudomonas.

7. The combination of claim 5, wherein the gram-positive bacteria is Staphylococcus aureus, methicillin-resistant Staphylococcus aureus, Streptococcus epidermidis, Streptococcus pyogenes, and the gram-negative bacteria is Pseudomonas aeruginosa.

8. The combination of claim 4, wherein the fungi is Candida.

9. The combination of claim 4, wherein the fungi is Candida albicans.

10. The combination of claim 1, wherein the photosensitizer is selected from the group consisting of hematoporphyrins 3,1-meso tetrakis (o-propionamidophenyl) porphyrin, hydroporphyrins, chlorin e6 monoethylendiamine monamide, the hematoporphyrin mixture Photofrin II, benzophorphyrin derivatives, tetracyanoethylene adducts, dimethyl acetylene dicarboxylate adducts, Diels-Adler adducts, a naphthalocyanine, toluidine blue O, aluminum sulfonated and disulfonated phthalocyanine ibid, a tetrasulfated derivative, sulfonated aluminum naphthalocyanines, methylene blue, nile blue; crystal violet; azure β chloride, toluidine blue, chlorine e6, and Rose Bengal.

11. The combination of claim 1, wherein the photosensitizer is selected from the group consisting of hematoporphyrin, methylene blue, toluidine blue, chlorine e6 and Rose Bengal.

12. The combination of claim 1, wherein the photosensitizer is in free, micellar or liposomal form.

13. The combination of claim 1, wherein the chitosan has the molecular weight ranging from 0.5 kDa to 1000 kDa.

14. The combination of claim 1, wherein the amount of chitosan is at least 0.001% (w/v).

15. The combination of claim 1, wherein the amount of chitosan ranges from 0.005% (w/v) to 0.025 (w/v), 0.005% (w/v) to 0.01 (w/v), 0.005% (w/v) to 0.25% (w/v), 0.005% (w/v) to 0.6% (w/v), 0.005% (w/v) to 1% (w/v) or 0.005% (w/v) to 5% (w/v).

16. The combination of claim 1, wherein the amount of chitosan ranges from 0.025 (w/v) to 0.01 (w/v), 0.01 (w/v) to 0.25% (w/v), 0.25% (w/v) to 0.6% (w/v), 0.6% (w/v) to 1% (w/v) or 1% (w/v) to 5% (w/v).

17. The combination of claim 1, which can be further formulated with pharmaceutically acceptable excipients.

18. The combination of claim 1, which has a synergistic effect in killing microorganisms.

19. A method of killing microorganisms in a subject which comprises the steps of administering the combination of claim 1 to the subject and irradiating the photosensitizer in the combination, thereby killing the microorganisms.

20. A method of treating microorganism infection in a subject which comprises the steps of administering the combination of claim 1 to the subject and irradiating the photosensitizer in the combination, thereby treating the microorganism infection.
